(19) United States
(12) Patent Application Publication
(10) Pub. No.: US 2008/0020472 A1
(43) Pub. Date: Jan. 24, 2008

(54) Method for detecting an inflammatory disease or cancer

(75) Inventors: Lian Shan, Broadview Heights, OH (US); Lorelei D. Davis, Shaker Heights, OH (US)

Correspondence Address: FRISHAUF, HOLTZ, GOODMAN & CHICK, PC, 220 Fifth Avenue, 16TH Floor, NEW YORK, NY, 10001-7708, US

(73) Assignee: FRANTZ BIOMARKERS, LLC, Mentor, OH (US)

(21) Appl. No.: 11/601,076

(22) Filed: Nov. 17, 2006

Related U.S. Application Data
(60) Provisional application No. 60/738,849, filed on Nov. 22, 2005.
(60) Provisional application No. 60/843,088, filed on Sep. 8, 2006.

(51) Int. Cl. G01N 33/92

(52) U.S. Cl. 436/64; 436/71

(57) ABSTRACT

A method of detecting an inflammatory disease or a cancer in a test subject comprising determining the amount of plasmenyl-PE or a biomarker having a mass charge ratio of approximately 698.2, 722.2, 726.2 or 750.2 in a sample of bodily fluid taken from the test subject and comparing the amount of plasmenyl-PE (or the biomarker) in the sample of the bodily fluid from the test subject to a range of amounts of plasmenyl-PE (or the biomarker) found in samples of the bodily fluid from a group of normal subjects of the same species as the test subject and lacking the inflammatory disease or the cancer, whereby a change in the amount of the plasmenyl-PE (or the biomarker) (such as a lower amount) in the sample of the bodily fluid from the test subject indicates the presence of the inflammatory disease or the cancer.

### CROSS-REFERENCE TO RELATED APPLICATIONS

This application claims the benefit under 35 USC 119(e) of provisional application Ser. No. 60/738,849 filed Nov. 22, 2005 and provisional application Ser. No. 60/843,088 filed Sep. 8, 2006, the entire contents of both of which provisional applications are incorporated by reference herein.

### BACKGROUND OF THE INVENTION

1. Field of the Invention

A method of detecting an inflammatory disease or cancer in a test subject. The present invention is further directed to a method for detecting the occurrence of ovulation during a menstrual cycle. More particularly, the present invention relates to a method for detecting inflammatory disease or cancer in a test subject by determining the amount of a plasmalogen, such as plasmenyl-PE (“PPE”), in a sample of bodily fluid taken from the test subject. The present invention is particularly useful as a screening test for cancer, such as ovarian cancer.

2. Background Information

Inflammation is the body's basic response to infection, irritation or trauma. The characteristic signs of an inflammatory response are redness, warmth, swelling and pain. The inflammatory reaction guides components in the immune system to the site of the trauma or infection, which can be observed as increased blood flow and vascular permeability, which, in turn, allows signal substances and white blood cells to leave the circulation.

Inflammation is thus a process that protects animals from invading pathogens. One mechanism of inflammatory action is the induction of oxidative pathways to damage the pathogens. Inflammation and oxidative stress have also been linked to non-pathogenic human diseases.

Chronic inflammatory diseases, such as rheumatoid arthritis, irritable bowel disease, systemic lupus erythematosus, multiple sclerosis and type-1 diabetes, affect more than 50 million Americans. Many of these diseases are debilitating and are becoming increasingly common in our society.

Follicular extrusion during ovulation exposes ovarian epithelial cells to conditions that induce inflammation and subsequent oxidative damage. Malfunctions in the repair of this oxidative damage can lead to ovarian cancer, a condition that causes further inflammation and oxidative stress. Plasmalogens are naturally occurring anti-oxidants; they are themselves oxidized in these processes, protecting other molecules from oxidation.

Ovarian cancer has been hypothesized to be caused by oxidative stress for the following reasons.

(a) Follicular extrusion during ovulation precipitates an inflammatory pathway that exposes ovarian epithelial cells to oxidative stress (Roberta B. Ness and Carrie Cottreau, “Possible Role of Ovarian Epithelial Inflammation in Ovarian Cancer,” Journal of National Cancer Institute, Vol. 91, No. 17, 1459-1467, Sep. 1, 1999).

(b) Cells surrounding the site of extrusion show elevated levels of DNA lesions that are indicators of oxidative stress, particularly 8-oxoguanine (W. J. Murdoch and J. F. Martinchick, “Oxidative Damage Due to DNA of Ovarian Cancer Surface Epithelial Cells Affected by Ovulation: Carcinogenic Implication and Chemoprevention,” Exp. Biol. Med., 229(6), 546-552, June 2004).

(c) Malfunctions in the repair of these DNA lesions are hypothesized to lead to metaplasia and carcinogenesis (W. J. Murdoch, “Metaplastic Potential of p53 Down-Regulated in Ovarian Surface Epithelial Cells Affected by Ovulation,” Cancer Lett., 191(1), 75-81, Feb. 28, 2003).

There is the following genetic evidence to support the above hypothesis.

(a) Women with mutations in the breast cancer BRCA 1 and 2 genes are at elevated risk for developing ovarian cancer. Cells that have mutations in the BRCA 1 and 2 genes are deficient in the repair of 8-oxoguanine lesions (F. LePage et al., “BRCA 1 and BRCA 2 are Necessary for the Transcription-Coupled Repair of the Oxidative 8-Oxoguanine Lesion in Human Cells,” Cancer Res., 60(19), 5548-5552, Oct. 1, 2000).

(b) Inhibition of the tumor suppressor p53 with anti-sense RNA prevents the repair of 8-oxoguanine DNA lesions in ovarian epithelial cells and results in the expression of CA-125, a marker for ovarian cancer (W. J. Murdoch, supra).

(c) Patients with ovarian cancer have a higher than normal rate of mutation in the gene of superoxide dismutase-2 (SOD-2), an enzyme necessary for repair of oxidative damage (S. H. Olson et al., “Genetic Variants in SOD2, MPO and NQ01, and Risk of Ovarian Cancer,” Gynecol. Oncol., 93(3), 615-620, June 2004).

There is also the following clinical evidence to support the above hypothesis.

(a) An elevated risk of ovarian cancer has long been associated with hyperovulation. Women who have fewer lifetime ovulations—late onset of menses, pregnancy, etc.—have a decreased risk of developing ovarian cancer (Ness et al., supra).

(b) The anti-oxidant vitamin E has been shown to lower the incidence of ovarian cancer in ewes (Murdoch and Martinchick, supra).

(c) Women who have rheumatoid arthritis and are on long-term anti-inflammatory, anti-oxidant medications have a lower incidence of ovarian cancer.

Ovarian tumor cells generate oxidative enzymes, as evidenced by the following:

(a) Ovarian carcinomas have elevated peroxidase activity (J. A. Holt et al., “Estrogen Receptor and Peroxidase Activity in Epithelial Ovarian Carcinomas,” J. Natl. Cancer Inst., 67(2), 307-318, August 1981).

(b) Patients with ovarian cancer show depleted serum concentrations of anti-oxidants, indicating oxidative stress (K. Senthil et al., “Evidence of Oxidative Stress in the Circulation of Ovarian Cancer Patients,” Clin. Chim. Acta., 339 (1-2), 27-31, January 2004; Ness et al, supra).

Plasmalogens are hypothesized to protect against oxidative stress (B. Engelmann, “Plasmalogen: Targets for Oxidants and Major Lipophilic Antioxidants,” Biochem. Soc. Trans., 32(Pt1), 147-150, February 2004).

(a) Plasmenyl-PE protects membrane lipids and cholesterol from oxidation (R. Maeba and N. Veta, “A Novel Antioxidant Action of Ethanolamine Plasmalogens in Lowering the Oxidizability of Membranes,” Biochemical Science Transactions, (2004), Vol. 32, Part 1, 2003).

(b) Deficiencies in plasmalogen synthesis cause human diseases that are associated with high levels of oxidized lipids.

(c) The presence of 16:0, 22:6 plasmenyl-PE in the brain has been hypothesized to be protective against oxidation (Yavin et al., Nutr. Neurosci., 5, 149-157 (2002)).

Decreased serum plasmalogen is associated with other diseases of oxidative stress for the following reasons:

(a) The serum concentrations of plasmalogen by-products—dimethyl acetals (DMAs)—are lower in patients who are undergoing hemodialysis, a treatment known to cause oxidative stress (T. Brosche et al., “Decreased Concentrations of Serum Phospholipid Plasmalogens Indicate Oxidative Burden of Uraemic Patients Undergoing Haemodialysis,” Nephron, Vol. 90, No. 1, 58-63, 2002).

(b) Peroxisomal disorders such as Zellweger's syndrome are deficient in plasmalogen synthesis and show hyper-oxidation of neural membrane lipids.

Ovarian cancer is one of the deadliest cancers for women, due to its high fatality rate. In the United States in 2005, it was estimated that 22,000 women would be diagnosed with ovarian cancer and 16,000 women would die of ovarian cancer. Unfortunately, heretofore, only 25% of ovarian cancer patients were diagnosed at stage I. Most of the patients were diagnosed at an advanced stage, stage III or IV, at which the 5-year survival rate decreases to 20 to 25% from 95% at stage I.

Presently, the most commonly used biomarker for diagnosing ovarian cancer is CA-125, a group of surface glycoproteins with uncertain biological function. Although CA-125 is elevated in 82% of women with advanced ovarian cancer, it has very limited clinical application for the detection of early stage disease, exhibiting a positive predictive value of less than 10%. The addition of physical examination by diagnostic ultrasound improves the positive predictive value to 20%, which is still too low to meet the requirement for cancer detection. Developing a clinical test to diagnose ovarian cancer with high sensitivity and specificity at the early stage has become the most urgent issue in battling this refractory disease.

Frequently, the detection of cancer depends upon the detection and inspection of a tumor mass, which has reached sufficient size to be detected by physical examination. The detection of molecular markers of carcinogenesis and tumor growth can solve many of the problems associated with the physical examination of tumors. Samples taken from the patient for screening by molecular techniques are typically blood or urine, and thus require minimally invasive techniques. Thus, they can be used on a regular basis to screen for cancers. In addition, because molecular markers may appear before the tumor reaches a detectable size, it is possible to detect cancers at very early stages in the progression of the disease.

Biomarkers identified from serum proteomic analysis for the detection of ovarian cancer are discussed in Z. Zhang et al., Cancer Research, 64, 5882-5890, Aug. 15, 2004.

Methods for detecting cancer associated with elevated concentrations of lysophospholipids have been described in US 2002/0123084 and US 2002/0150955.

U.S. Pat. No. 6,500,633 discloses a method of detecting carcinomas by measuring the level of a glycerol compound, such as glycerol-3-phosphate, in a plasma, serum or urine specimen from a patient.

### SUMMARY OF THE INVENTION

It is an object of the present invention to provide a non-invasive method for detecting an inflammatory disease in a test subject.

It is also an object of the present invention to provide a non-invasive method for detecting a cancer in a test subject.

It is another object of the present invention to provide a non-invasive method of detecting a gynecologic cancer, such as ovarian cancer, in a test subject.

It is a further object of the present invention to utilize a molecular marker for the screening and diagnosis of an inflammatory disease or a cancer, such as ovarian cancer.

It is a still further object of the present invention to provide a non-invasive method to detect the occurrence of ovulation during a menstrual cycle.

It is another object of the present invention to provide a non-invasive method to monitor the presence of an inflammatory disease or a cancer over time.

The above objects, as well as other objects, advantages and aims are satisfied by the present invention.

The present invention concerns a method of detecting an inflammatory disease in a test subject comprising:

(a) determining the amount of plasmalogen, such as plasmenyl-PE, in a sample of a bodily fluid taken from the test subject, and

(b) comparing the amount of plasmalogen, such as plasmenyl-PE, in the sample of bodily fluid taken from the test subject to a range of amounts of plasmalogen, such as plasmenyl-PE found in samples of the bodily fluid taken from a group of normal subjects of the same species as the test subject and lacking the inflammatory disease (e.g., if the bodily fluid taken from the test subject is serum, then the bodily fluid taken from each member of the group of normal subjects will also be serum), whereby a change in the amount (such as a lower amount) of the plasmalogen, such as plasmenyl-PE, in the sample of the bodily fluid taken from the test subject indicates the presence of the inflammatory disease.

The present invention also further relates to a method of detecting an inflammatory disease in a test subject comprising:

(a) determining the amount of a biomarker having a mass charge ratio of approximately 698.2, 722.2, 726.2 or 750.2 in a sample of a bodily fluid taken from the test subject, and

(b) comparing the amount of the biomarker in the sample of the bodily fluid taken from the test subject to a range of amounts of the biomarker found in samples of the bodily fluid taken from a group of normal subjects of the same species as the test subject and lacking the inflammatory disease (e.g., if the bodily fluid taken from the test subject is serum, then the bodily fluid taken from each member of the group of normal subjects will also be serum), whereby a change in the amount (such as a lower amount) of the biomarker in the sample of the bodily fluid taken from the test subject indicates the presence of the inflammatory disease.

The present invention further concerns a method of detecting a cancer (such as ovarian cancer) in a test subject comprising:

(a) determining the amount of plasmalogen, such as plasmenyl-PE, in a sample of a bodily fluid taken from the test subject, and

(b) comparing the amount of plasmalogen, such as plasmenyl-PE, in the sample of the bodily fluid taken from the test subject to a range of amounts of plasmalogen, such as plasmenyl-PE found in samples of the bodily fluid taken from a group of normal subjects of the same species as the test subject and lacking the cancer (e.g., if the bodily fluid taken from the test subject is serum, then the bodily fluid taken from each member of the group of normal subjects will also be serum), whereby a change in the amount (such as a lower amount) of the plasmalogen, such as plasmenyl-PE, in the sample of the bodily fluid taken from the test subject indicates the presence of the cancer, wherein when the cancer is ovarian cancer, the plasmalogen is not plasmenyl-PA (“PPA”) or plasmenyl-PC (“PPC”), and wherein when the cancer is breast cancer, the plasmalogen is not PPE or PPA.

The present invention also relates to a method of detecting a cancer in a test subject comprising:

(a) determining the amount of a biomarker having a mass charge ratio of approximately 698.2, 722.2, 726.2 or 750.2 in a sample of a bodily fluid taken from the test subject, and

(b) comparing the amount of the biomarker in the sample of the bodily fluid taken from the test subject to the biomarker found in samples of the bodily fluid taken from a group of normal subjects of the same species as the test subject and lacking the cancer (e.g., if the bodily fluid taken from the test subject is serum, then the bodily fluid taken from each member of the group of normal subjects will also be serum), whereby a change in the amount (such as a lower amount) of the biomarker in the sample of the bodily fluid taken from the test subject indicates the presence of the cancer.

The present invention is also directed to a method of detecting the occurrence of ovulation during a menstrual cycle in a test subject comprising:

(a) determining the amount of plasmalogen, such as plasmenyl-PE, in a sample of a bodily fluid taken from a female test subject, and

(b) comparing the amount of plasmalogen, such as plasmenyl-PE, in the sample of the bodily fluid taken from the female test subject to a range of amounts of plasmalogen, such as plasmenyl-PE found in samples of the bodily fluid taken from a group of non-ovulating female subjects of the same species as the test subject (e.g., if the bodily fluid taken from the female test subject is serum, then the bodily fluid taken from each member of the group of non-ovulating female subjects will also be serum), whereby a change in the amount (such as a lower amount) of the plasmalogen, such as plasmenyl-PE in the sample of the bodily fluid taken from the female test subject indicates the occurrence of ovulation.

The present invention is further directed to a method of detecting the occurrence of ovulation during a menstrual cycle in a female test subject comprising:

(a) determining the amount of a biomarker having a mass charge ratio of approximately 698.2, 722.2, 726.2 or 750.2 in a sample of a bodily fluid taken from the female test subject, and

(b) comparing the amount of the biomarker in the sample of the bodily fluid taken from the female test subject to the biomarker found in samples of the bodily fluid taken from a group of non-ovulating female subjects of the same species as the test subject (e.g., if the bodily fluid taken from the female test subject is serum, then the bodily fluid taken from each member of the group of non-ovulating female test subjects will also be serum), whereby a change in the amount (such as a lower amount) of the biomarker in the sample of the bodily fluid taken from the test subject indicates the occurrence of ovulation.

The present invention is also directed to a method for monitoring an inflammatory disease in a test subject over time comprising:
(a) determining the amount of plasmalogen, such as plasmenyl-PE, in a sample of a bodily fluid taken from the test subject at a first time,
(b) determining the amount of plasmalogen, such as plasmenyl-PE, in a sample of the bodily fluid taken from said test subject at a second time (e.g., if the bodily fluid taken in step (a) is serum, then the bodily fluid taken in step (b) will also be serum), which is later than the first time,
(c) comparing the amounts of plasmalogen, such as plasmenyl-PE, in step (a) and step (b) to determine whether there has been an increase or a decrease in the amount of plasmalogen, such as plasmenyl-PE, in the sample of the bodily fluid taken from the test subject at the later time relative to the amount of the plasmalogen, such as plasmenyl-PE, in the sample taken from the test subject at the first time, whereby a decrease in the amount of the plasmalogen, such as plasmenyl-PE, in the sample of the bodily fluid at the later time indicates the presence of, or worsening of, the inflammatory disease, or an increase in the amount of the plasmalogen, such as plasmenyl-PE, in the sample of the bodily fluid at the later time indicates an absence, or improvement of, the inflammatory disease.

The present invention further relates to a method for monitoring an inflammatory disease in a test subject over time comprising:
(a) determining the amount of a biomarker having a mass charge ratio of approximately 698.2, 722.2, 726.2 or 750.2 in a sample of a bodily fluid taken from the test subject at a first time,
(b) determining the amount of the biomarker in a sample of the bodily fluid taken from a test subject at a second time (e.g., if the bodily fluid in step (a) is serum, then the bodily fluid in step (b) will also be serum), which is later than a first time,
(c) comparing the amounts of the biomarker in step (a) and step (b) to determine whether there has been an increase or a decrease in the amount of the biomarker in the sample of the bodily fluid taken from the test subject at the later time relative to the amount of the biomarker in the sample of the bodily fluid taken from the test subject at the first time, whereby a decrease in the amount of the biomarker in the sample of the bodily fluid at the later time indicates the presence of, or worsening of, the inflammatory disease, or an increase in the amount of the biomarker in the sample of the bodily fluid at the later time indicates an absence, or improvement of, the inflammatory disease.

The present invention further concerns a method for monitoring a cancer in a test subject over time comprising:
(a) determining the amount of plasmalogen, such as plasmenyl-PE, in a sample of a bodily fluid taken from the test subject at a first time,
(b) determining the amount of plasmalogen, such as plasmenyl-PE, in a sample of the bodily fluid taken from said test subject at a second time (e.g., if the bodily fluid in step (a) is serum, then the bodily fluid in step (b) will also be serum), which is later than the first time,
(c) comparing the amounts of plasmalogen, such as plasmenyl-PE, in step (a) and step (b) to determine whether there has been an increase or a decrease in the amount of the plasmalogen, such as plasmenyl-PE, in the sample of the bodily fluid taken from the test subject at the later time relative to the amount of the plasmalogen, such as plasmenyl-PE, in the sample of the bodily fluid taken from the test subject at the first time, whereby a decrease in the amount of the plasmalogen, such as plasmenyl-PE, in the sample of the bodily fluid taken from the test subject at the later time indicates the presence of, or worsening of, the cancer, or an increase in the amount of the plasmalogen, such as plasmenyl-PE, in the sample of the bodily fluid at the later time indicates an absence, or improvement of, the cancer,

wherein, when the cancer is ovarian cancer, the plasmalogen is not PPA or PPC, and wherein, when the cancer is breast cancer, the plasmalogen is not PPE or PPA.

The present invention also relates to a method for monitoring a cancer in a test subject over time comprising:
(a) determining the amount of a biomarker having a mass charge ratio of approximately 698.2, 722.2, 726.2 or 750.2 in a sample of a bodily fluid taken from the test subject at a first time,
(b) determining the amount of the biomarker in a sample of the bodily fluid taken from said test subject at a second time (e.g., if the bodily fluid taken in step (a) is serum, then the bodily fluid in step (b) will also be serum), which is later than the first time,
(c) comparing the amounts of the biomarker in step (a) and step (b) to determine whether there has been an increase or a decrease in the amount of the biomarker in the sample of the bodily fluid taken from the test subject at the later time relative to the amount of the biomarker in the sample of the bodily fluid taken from the test subject at the first time, whereby a decrease in the amount of the biomarker in the sample of the bodily fluid taken from the test subject at the later time indicates the presence of, or worsening of, the cancer, or an increase in the amount of the biomarker in the sample of the bodily fluid at the later time indicates an absence, or improvement of, the cancer.

In all of the above-described methods (such as detecting an inflammatory disease, detecting a cancer, detecting the occurrence of ovulation, monitoring an inflammatory disease and monitoring a cancer), instead of determining the amount of plasmalogen, such as plasmenyl-PE, the amount of products of plasmalogen oxidation, such as products of plasmenyl-PE oxidation (such as dimethyl acetals) can be determined. For example, the presence of ovarian cancer can be detected by measuring a decrease in serum concentrations of plasmalogen, such as plasmenyl-PE, or an increase in the products of plasmalogen oxidation, such as plasmenyl-PE, oxidation. Similarly, the occurrence of ovulation during a menstrual cycle can be detected by a decrease in the serum concentration of plasmalogen, such as plasmenyl-PE or an increase in the products of plasmalogen oxidation, such as plasmenyl-PE oxidation.

### BRIEF DESCRIPTION OF THE DRAWINGS

FIG. 1 is a graphical representation of the levels of 18:0, 22:6 PPE in the following serum samples: early stage ovarian (“ov”) cancer, advanced stage ov cancer and healthy control.

FIG. 2 is a graphical representation of the levels of 18:0, 20:4 PPE in the following serum samples: early stage ov cancer, advanced stage ov cancer and healthy control.

FIG. 3 is a graphical representation of the levels of 18:0, 18:1 PPE in the following serum samples: early stage ov cancer, advanced stage ov cancer and healthy control.

FIG. 4 is a graphical representation of the levels of 18:0, 18:2 PPE in the following serum samples: early stage ov cancer, advanced stage ov cancer and healthy control.

FIG. 5 is a graphical representation of the levels of 16:0, 22:6 PPE in the following serum samples: early stage ov cancer, advanced stage ov cancer and healthy control.

FIG. 6 is a graphical representation of the levels of 16:0, 20:4 PPE in the following serum samples: early stage ov cancer, advanced stage ov cancer and healthy control.

FIG. 7 is a graphical representation of the levels of 16:0, 18:1 PPE in the following serum samples: early stage ov cancer, advanced stage ov cancer and healthy control.

FIG. 8 is a graphical representation of the levels of 16:0, 18:2 PPE in the following serum samples: early stage ov cancer, advanced stage ov cancer and healthy control.

FIG. 9 is a graphical representation of the levels of 18:0, 22:6 PPE in the following plasma samples: pre- or intra-surgery ov cancer, post-surgery ov cancer, pre-surgery breast cancer, benign gynecological disease (“BYN”) control, high-risk control and healthy control.

FIG. 10 is a graphical representation of the levels of 18:0, 20:4 PPE in the following plasma samples: pre- or intra-surgery ov cancer, post-surgery ov cancer, pre-surgery breast cancer, benign gynecological disease (“BYN”) control, high-risk control and healthy control.

FIG. 11 is a graphical representation of the levels of 18:0, 18:1 PPE in the following plasma samples: pre- or intra-surgery ov cancer, post-surgery ov cancer, pre-surgery breast cancer, benign gynecological disease (“BYN”) control, high-risk control and healthy control.

FIG. 12 is a graphical representation of the levels of 18:0, 18:2 PPE in the following plasma samples: pre- or intra-surgery ov cancer, post-surgery ov cancer, pre-surgery breast cancer, benign gynecological disease (“BYN”) control, high-risk control and healthy control.

FIG. 13 is a graphical representation of the levels of 16:0, 22:6 PPE in the following plasma samples: pre- or intra-surgery ov cancer, post-surgery ov cancer, pre-surgery breast cancer, benign gynecological disease (“BYN”) control, high-risk control and healthy control.

FIG. 14 is a graphical representation of the levels of 16:0, 20:4 PPE in the following plasma samples: pre- or intra-surgery ov cancer, post-surgery ov cancer, pre-surgery breast cancer, benign gynecological disease (“BYN”) control, high-risk control and healthy control.

FIG. 15 is a graphical representation of the levels of 16:0, 18:1 PPE in the following plasma samples: pre- or intra-surgery ov cancer, post-surgery ov cancer, pre-surgery breast cancer, benign gynecological disease (“BYN”) control, high-risk control and healthy control.

FIG. 16 is a graphical representation of the levels of 16:0, 18:2 PPE in the following plasma samples: pre- or intra-surgery ov cancer, post-surgery ov cancer, pre-surgery breast cancer, benign gynecological disease (“BYN”) control, high-risk control and healthy control.

### DETAILED DESCRIPTION OF THE INVENTION

Plasmalogens are a class of phospholipids characterized by the presence of a vinyl-ether bond present at the sn-1 position of the glycerol backbone, rather than an ester bond as in diacylglycerophospholipids. The sn-2 position is occupied by a fatty acid. Two kinds of plasmalogens have been reported as being present in biological samples, namely ethanolamine plasmalogen (also called plasmenyl-PE, wherein an ethanolamine group is attached to the sn-3 phosphate group) and choline plasmalogen (also called plasmenyl-PC (“PPC”), a choline group is attached to the sn-3 phosphate group). Brain myelin possesses the highest content of plasmenyl-PE (almost exclusively as the version with docosahexaenoic acid, 22:6, as a fatty acid), whereas the heart muscle has a higher content of plasmenyl-PC.

Lowered levels of 16:0, 22:6 plasmenyl-PE (“pl-PE” or “PPE”) have been associated with neurological peroxisomal disorders such as Zellweger's syndrome.

Moderate amounts of plasmalogens are found in the kidneys, skeletal muscles, the spleen and blood cells. The biological functions of plasmalogens are not clear. It is considered that plasmalogens play the following roles in the human body: preventing oxidation, mediating membrane dynamics, acting as storage depots of fatty acids and serving as lipid mediators.

Plasmenyl-PA (phosphatidic acid plasmalogen) (“pl-PA” or “PPA”) is a class of plasmalogen with a phosphatidic acid group attached to the sn-3 position of the glycerol backbone. Its structure is close to the structures of the other two kinds of plasmalogens, except that the sn-3 phosphate group is not esterified to a choline or an ethanolamine group.

The structures of plasmenyl-PE, plasmenyl-PC and plasmenyl-PA are as follows: 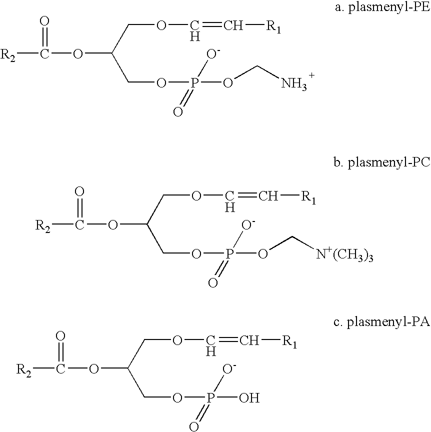
In the plasmalogen structures, R₁ and R₂ are alkyl chains. Another plasmalogen is plasmenyl-PI. The structure of plasmenyl-PI is as follows:

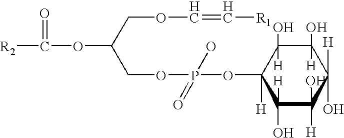

Plasmalogens, such as plasmenyl-PE compounds, that can be used in the methods discussed herein can include any combination of the following ratios of number of carbon atoms to number of double bonds connecting the carbon atoms: at the sn-1 position, 12:1, 14:1, 16:1, 16:2, 18:1, 18:2, 18:3, 18:4, 20:1, 20:5, 22:1 and 22:7; at the sn-2 position, 12:0, 14:0, 16:0, 16:1, 18:0, 18:1, 18:2, 18:3, 20:0, 20:4, 22:0 and 22:6. In the names of the plasmalogens that were used, one of the double bonds in the sn-1 position is the vinyl ethyl bond which is not considered to be part of the fatty acid chain.

Non-limiting examples of plasmenyl-PE compounds which are sought to be detected in the methods disclosed herein include the following:

18:0, 22:6 PPE,

18:0, 20:4 PPE (mass charge ratio of approximately 750.2)

16:0, 22:6 PPE,

18:0, 18:1 PPE,

18:0, 18:2 PPE, (mass charge ratio of approximately 726.2),

16:0, 20:4 PPE, (mass charge ratio of approximately 722.2),

16:0, 18:1 PPE and

16:0, 18:2 PPE (mass charge ratio of approximately 698.2).

For detecting ovarian cancer, the preferred markers are 18:0, 18:2 PPE, 18:0, 20.4 PPE, 16:0, 18:2 PPE and 16:0, 20:4 PPE.

Non-limiting examples of plasmenyl-PA compounds which are sought to be detected in the methods disclosed herein include the following:

16:0, 18:2 PPA,

16:0, 20:4 PPA,

16:0, 22:6 PPA and

16:0,-18:1 PPA.

A preferred plasmenyl-PA compound is 16:0, 18:2 plasmenyl-PA, which has a mass charge ratio of approximately 655.3.

In an embodiment of the invention, the amount of plasmalogen, such as plasmenyl-PE or a biomarker having a mass charge ratio of approximately 655.3, 698.2, 722.2, 726.2 or 750.2 found in a sample of a bodily fluid taken from a test subject, is compared to the amount of plasmalogen, such as plasmenyl-PE, or the biomarker having a mass charge ratio of approximately 655.3, 698.2, 722.2, 726.2 or 750.2, found in a sample from a normal subject of the same species as the test subject lacking the cancer (e.g., if the test subject is a human, then the normal subject is a human who does not have the cancer). A lower amount of the plasmalogen, such as plasmenyl-PE, or the biomarker having a mass charge ratio of approximately 655.3, 698.2, 722.2, 726.2 or 750.2 found in the sample of the bodily fluid, from the test subject when compared to the amount of the plasmalogen, such as plasmenyl-PE or the biomarker having a mass charge ratio of approximately 698.2, 722.2, 726.2 or 750.2 in the sample of the bodily fluid taken from the normal subject, indicates the presence of the cancer.

The term “approximately 655.3, 698.2, 722.2, 726.2 or 750.2” used herein means a mass charge ratio of 655.3, 698.2, 722.2, 726.2 or 750.2 or a mass charge ratio close to 655.3, 698.2, 722.2, 726.2 or 750.2.

The amount of plasmenyl-PE or the biomarker having a mass charge ratio of approximately 655.3, 698.2, 722.2, 726.2 or 750.2 detected in the sample taken from a test subject may be measured by first extracting lipids as described in detail infra. The amount of plasmenyl-PE or the biomarker having a mass charge ratio of approximately 655.3, 698.2, 722.2, 726.2 or 750.2 is then quantified using standard procedures, such as mass spectroscopy, gas chromatography, HPLC, NMR or other approaches.

In addition to the direct measurement of the plasmalogen, such as plasmenyl-PE or the biomarker having a mass charge ratio of approximately 655.3, 698.2, 722.2, 726.2 or 750.2, by extraction, antibodies, such as monoclonal antibodies reactive with a plasmalogen, such as plasmenyl-PE, or the biomarker can be used in an assay to detect the amount of plasmalogen, such as plasmenyl-PE, or the biomarker in a test sample. For example, anti-plasmalogen, such as anti-plasmenyl-PE (or anti-biomarker) antibodies may be labeled using standard procedures and used in assays including radioimmunoassays (RIA), both solid and liquid phase, fluorescence-linked assays or enzyme-linked immunosorbent assays (ELISA), wherein the antibody is used to detect the presence and amount of the plasmalogen, such as plasmenyl-PE (or the biomarker having a mass charge ratio of approximately 655.3, 698.2, 722.2, 726.2 or 750.2), in the fluid sample.

As discussed hereinabove, in the above-described methods, instead of determining the amount of plasmalogen, such as plasmenyl-PE, the amount of products of plasmalogen oxidation, such as plasmenyl-PE oxidation (such as dimethyl acetals) can be determined.

The oxidation products depend on the fatty acids in the plasmalogen, such as plasmenyl-PE, i.e., the double bonds in the unsaturated fatty acids are also targets for oxidation.

The following list sets forth oxidation products of a plasmenyl-PA with a 16:0 chain at the sn-1 position:
Oxidation occurring at the sn-1 position: LPA (1-lyso-2-R-sn-glycero-3-phosphatidic acid) 1-formyl-2-R-sn-glycero-3-phosphatidic acid
Oxidation occurring at the sn-2 position: 16:0p/4:0al-GPA (just for docosahexaenoic acid) 16:0p/6:1al-GPA
   16:0p/8:2al-GPA
   16:0p/9:2al-GPA
   16:0p/11:3al-GPA
   16:0p/12:3al-GPA
   16:0p/14:4al-GPA
   16:0p/15:4al-GPA
   16:0p/18:5al-GPA
   16:0p/4-hydroxy-7-oxo-hept-5-enoyl-GPA
   16:0p/7-hydroxy-10-oxo-dec-4,8-dienoyl-GPA
   16:0p/10-hydroxy-13-oxo-tridec-4,7,11-trienoyl-GPA
   16:0p/4-hydroxy-docosahexaenoyl-GPA
   16:0p/7-hydroxy-docosahexaenoyl-GPA
   16:0p/8-hydroxy-docosahexaenoyl-GPA
   16:0p/10-hydroxy-docosahexaenoyl-GPA
   16:0p/11-hydroxy-docosahexaenoyl-GPA
   16:0p/13-hydroxy-docosahexaenoyl-GPA
   16:0p/14-hydroxy-docosahexaenoyl-GPA
   16:0p/16-hydroxy-docosahexaenoyl-GPA
   16:0p/17-hydroxy-docosahexaenoyl-GPA
   16:0p/20-hydroxy-docosahexaenoyl-GPA
   16:0p/4-hydroperoxy-docosahexaenoyl-GPA
   16:0p/8-hydroperoxy-docosahexaenoyl-GPA
   16:0p/10-hydroperoxy-docosahexaenoyl-GPA
   16:0p/16-hydroperoxy-docosahexaenoyl-GPA
   16:0p/20-hydroperoxy-docosahexaenoyl-GPA

In the above list, “R” means a fatty acid group esterified to the sn-2 position of the glycerol backbone, “GPA” means glycerol phosphatidic acid, “p” means plasmalogen and “al” is aldehyde.

Oxidative products of plasmenyl-PC are discussed in Karin A. Zemski Berry et al., “Free Radical Oxidation of Plasmalogen Glycerophosphocholine Containing Esterified Docosahexaenoic Acid: Structure Determined by Mass Spectrometry,” Antioxidants & Redox Signaling, Vol. 7, No. 1-2, 157-169, January 2005. This publication reported that the oxidized phospholipid products resulting from the exposure of 1-0-hexadec-1′-enyl-2-docosahexaenoyl-sn-glycero-3-phosphocholine (16:0p/22:6-GPCho) to the free radical initiator 2,2′-azobis (2-amidinopropane)hydrochloride were examined. The radical-induced peroxidation of 16:0p/22:6-GPCho revealed two major classes of oxidized phospholipids. The first class of products was formed by oxidation at the sn-1 position and included 1-lyso-2-docosahexaenoyl-GPCho and 1-formyl-2-docosahexaenoyl-GPCho. Additionally, the second class of oxidized products where oxidation occurred at the sn-2 position, was classified into three categories that included chain-shortened ω-aldehydes, terminal γ-hydroxy-α, β-unsaturated aldehydes, and the addition of one or two oxygen atoms onto the sn-2 position of 16:0p/22:6-GPCho.

The amount of such oxidation products can also be determined by the techniques described herein with respect to determining the amount of plasmenyl-PE, but the parent-to-daughter-ion transition will be different. Thus, the amount of oxidation products can be determined using a MRM (multiple reaction monitoring) LC/ESI/MS/MS (liquid chromatography/electrospray ionization/tandem mass spectroscopy).

The test subject can be an eukaryotic organism, preferably a vertebrate, including, but not limited to, a mammal, a bird, a fish, an amphibian or a reptile. Preferably, the subject is a mammal, most preferably a human. The bodily fluid includes, but is not limited to, plasma, serum, urine, saliva, ascites, cerebral spinal fluid or pleural fluid. Preferably, the bodily fluid is plasma or serum which is obtained from a whole blood specimen from the test subject.

Methods disclosed herein can also be used to detect or screen for an inflammatory disease, such as arthritis (such as rheumatoid arthritis), inflammation of the heart (myocarditis), atherosclerosis, inflammation of the kidneys (nephritis), colitis, Crohn's disease, gastritis, multiple sclerosis, chronic obstructive pulmonary disease (“COPD”), thyroiditis, systemic lupus erythematosus, type 1 diabetes, psoriasis, meningitis, encephalitis, vasculitis, allergic rhinitis, atopic dermatitis, prostatitis, pelvic inflammatory disease, anklosing spondylitis, asthma, bronchitis, bursitis, tendonitis, Hodgkins's disease, rheumatic fever, myasthenia gravis, Behcet's syndrome, sarcoidosis, polymyositis, conjunctivitis, gingivitis, periarteritis nodosa and aplastic anemia.

Methods disclosed herein can be used to detect or screen for a broad range of cancers at an early stage. Such cancers include gynecological cancers, including ovarian cancer, breast cancer, cervical cancer, uterine cancer, endometrial cancer, peritoneal cancer, fallopian tube cancer and vulva cancer. Other cancers that can be detected according to the present invention include, but are not limited to, testicular cancer, colon cancer, lung cancer, prostate cancer, bladder cancer, kidney cancer, thyroid cancer, stomach cancer, pancreatic cancer, brain cancer, liver cancer, ureter cancer, esophageal cancer and larynx cancer. The present invention is preferably directed to detecting ovarian cancer.

Applicants have concluded that there is no correlation between determining amounts of PPE or PPA and the detection of breast cancer. Applicants have also concluded that there is no correlation between determining amounts of PPC and the detection of ovarian cancer. A patent application directed to methods for detecting ovarian cancer by detecting amounts of PPA is being filed concomitantly herewith, which names one of the co-inventors of this application.

The methods disclosed herein are non-invasive and require only a bodily fluid specimen, such as a blood specimen from the test subject (patient). Thus, such methods are useful for screening patients who have not been previously diagnosed as having an inflammatory disease, or carrying carcinoma, particularly patients who are at risk for carcinomas, especially ovarian carcinoma. Such patients include women at elevated risk by virtue of a family history of the disease, premenopausal women with anovulatory cycles, and postmenopausal women. The methods disclosed herein include a screening test for identifying within a risk population, a subset population with a greater propensity for developing an inflammatory disease or a cancer.

The methods disclosed herein can provide a number of benefits. First, the methods provide a rapid and economical screen for large numbers of subjects to promote early diagnosis of an inflammatory disease or a cancer, which can result in improved quality of life and better survival rates for patients.

Using the methods disclosed herein for prognosis, the medical professional can determine whether a subject with an inflammatory disease or a cancer in the early stages requires therapy or does not require therapy. This could also identify subjects who may not benefit from a particular form of therapy, e.g., surgery, chemotherapy, radiation or biological therapies. Such information could result in an improved therapy design for obtaining better responses to therapy.

Methods disclosed herein can also be used to identify patients for whom therapy should be altered from one therapeutic agent to another. This could obviate the need for “second look” invasive procedures to determine the patient's response to the therapy and facilitate decisions as to whether the particular type of therapy should be continued, terminated or altered.

Because cancers may recur in a significant number of patients with advanced cancers, early detection and continued monitoring over time using the methods of the present invention could identify early occult (i.e., “hidden”) recurrences prior to symptoms presenting themselves.

In addition, methods disclosed herein will facilitate distinguishing benign from malignant tumors. Masses in an organ such as the ovary can be initially detected using procedures such as ultrasound or by physical examination. Thereafter, methods disclosed herein can be used to diagnose the presence of cancer. This could obviate the need for surgical intervention, and/or identify subjects where continued monitoring is appropriate resulting in improved early detection and survival for cancer patients.

Yet another use for the methods disclosed herein is to determine the origin of an unknown primary tumor. The tissue of origin of malignant tumors in some parts of the body frequently cannot be determined using conventional techniques.

### EXAMPLES

The present invention will now be described in the context of the following non-limiting examples.

### Example 1

### Plasmenyl-PE in Serum Samples

Materials

18:0, 22:6 PPE; 18:0, 20:4 PPE; and 18:0, 18:1 PPE were purchased from Avanti Polar Lipids (Alabaster, Ala., USA). Using these lipids, it was determined that the MRM transition of 18:0, 22:6 PPE; 18:0, 20:4 PPE; 18:0, 18:1 PPE; 18:0, 18:2 PPE; 16:0 22:6 PPE; 16:0, 20:4 PPE; 16:0, 18:1 PPE; and 16:0, 18:2 PPE were 774.2→327.2, 750.2→303.2, 728.2→281.2, 726.2→279.2, 746.2→327.2, 722.2→303.2, 700.2→281.2, and 698.2→279.2 respectively.

### Example 1(a)

### Extraction of Plasmenyl-PE from Serum Samples

Lipid extraction was done according to the following procedure: Add 50 μL 10 μM 1,2-diheptadecanoyl-sn-glycerol-3-phosphoethanolamine, the internal standard for the assay, into 50 μl serum samples. Vortex and add 2 ml 2:1 methanol-chloroform into the samples. Vortex again and centrifuge the mixture for 5 minutes at 4000 rpm and 10° C. Transfer the upper liquid layer into a test tube and dry the liquid layer under nitrogen. Then add 400 μl 0.1 M ammonium acetate in methanol into the nitrogen-dried lipids. Vortex and transfer everything in the test tube into a microcentrifuge tube. Centrifuge at 9000 rpm for 5 minutes. Transfer the supernatant into an injection vial for LC/ESI/MS/MS analysis.

### Example 1(b)

### MRM LC/ESI/MS/MS Analysis for Plasmenyl-PE

LC/ESI/MS/MS analysis of plasmenyl-PE species was performed using a Quatro micro mass spectrometer (Micromass, Altrincham, U.K.) equipped with an electrospray ionization (ESI) probe and interfaced with a Shimadzu SCL-10AvpHPLC system (Shimadzu, Tokyo, Japan). Lipids were separated with a Betabasic-18 column (20×2.1 mm, 5 μm, Thermo Electron, Waltham, Mass.), protected by a Betabasic 18 pre-column (10×2.1 mm, 5 μm, Thermo Electron, Waltham, Mass.). 300 μl ammonium phosphate, pH=5.46 buffer was used as mobile phase A, while 9:1 (v:v) methanol-acetonitrile was used as mobile phase B. The gradient used was as follows: the column was first equilibrated with 70% B (30% A), followed by a linear change from 70% B (30% A) to 100% B (0% A) at 200 μl/minutes in the first 5 minutes. The gradient was kept at 100% in the following 3 minutes. Then it was changed back to 70% B (30% A) to re-equilibrate the column. The flow rate is 200 μl/minutes. Mass spectrometric analyses were performed online using electrospray ionization/tandem mass spectrometry in the negative multiple reaction monitoring (MRM) mode (capillary voltage: 3.5 KV, cone potential 55 V, collision energy 30 eV). The MRM transitions used have been described above in the section entitled “Materials” for Example 1.

### Example 1(c)

### Samples and Statistical Analysis

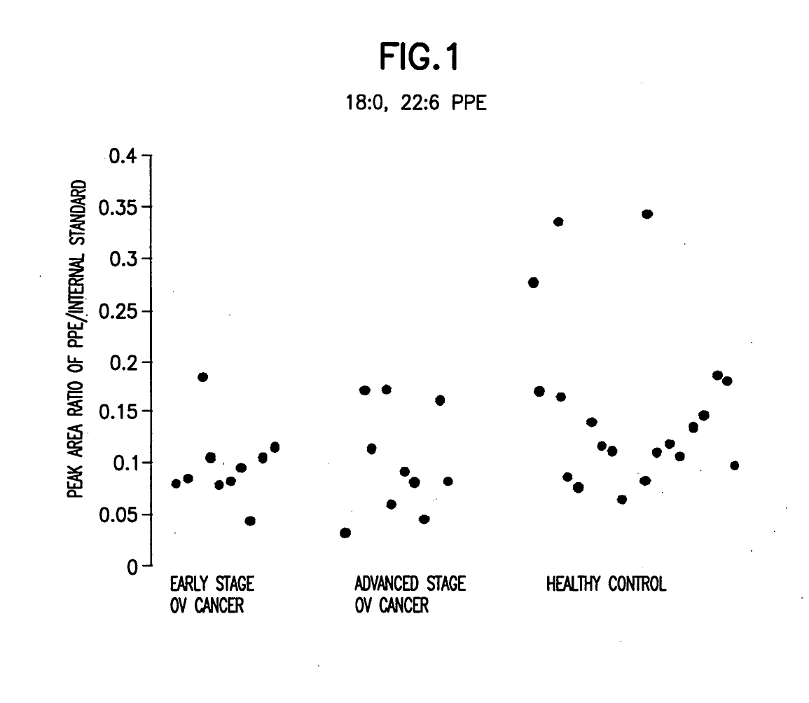

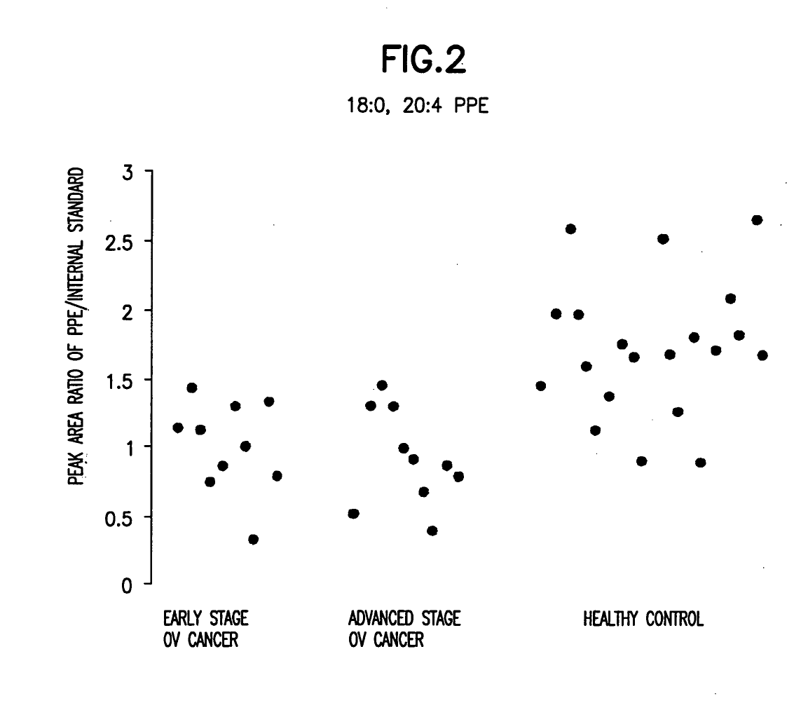

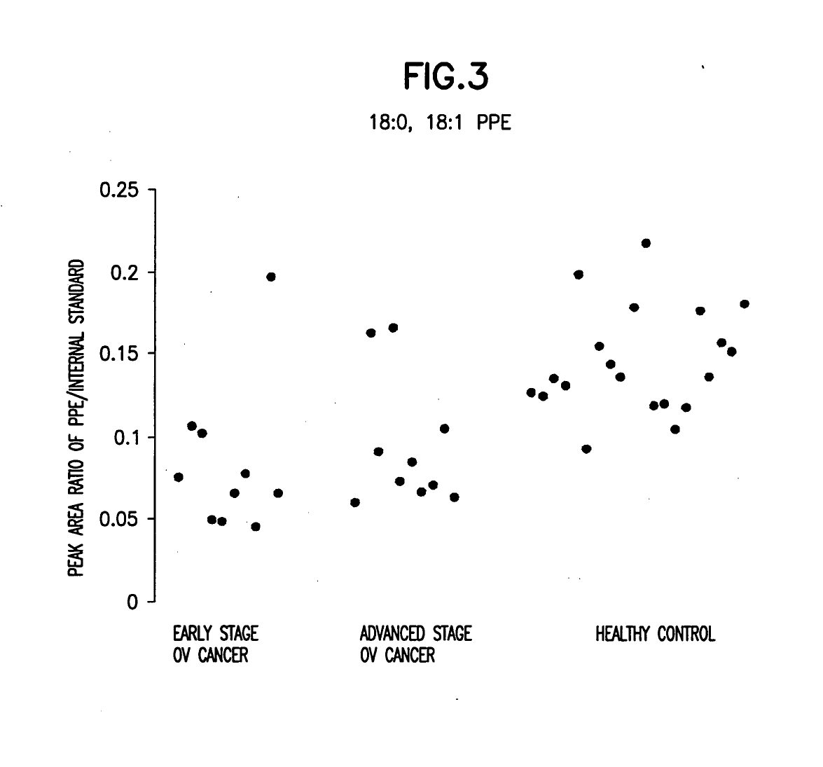

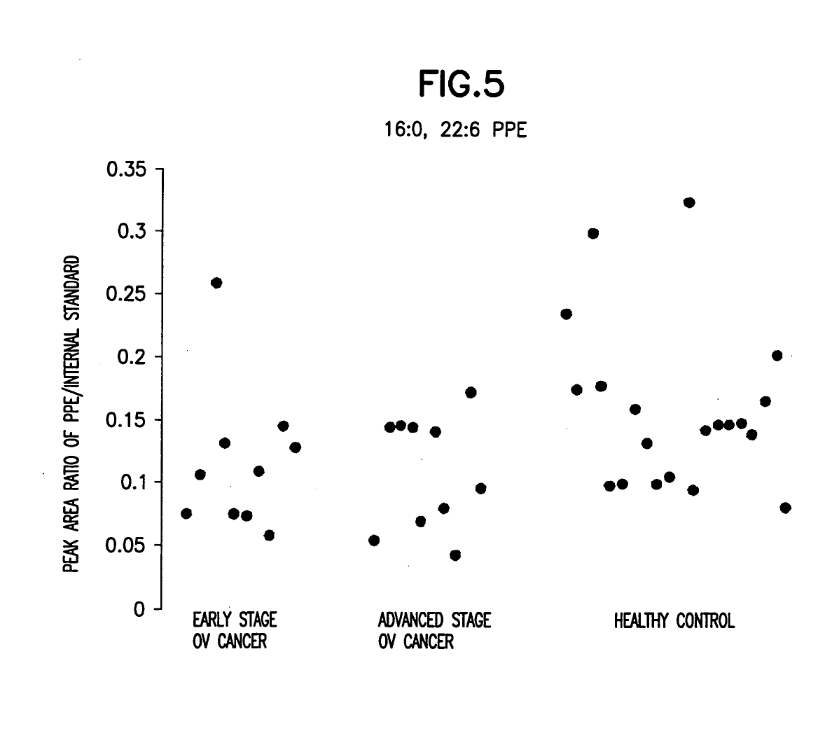

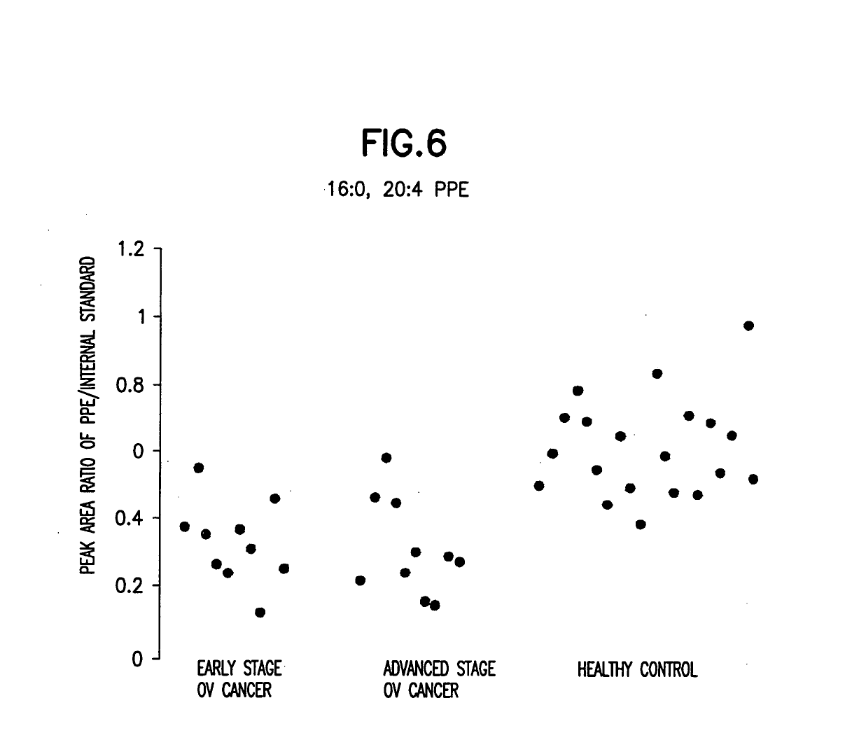

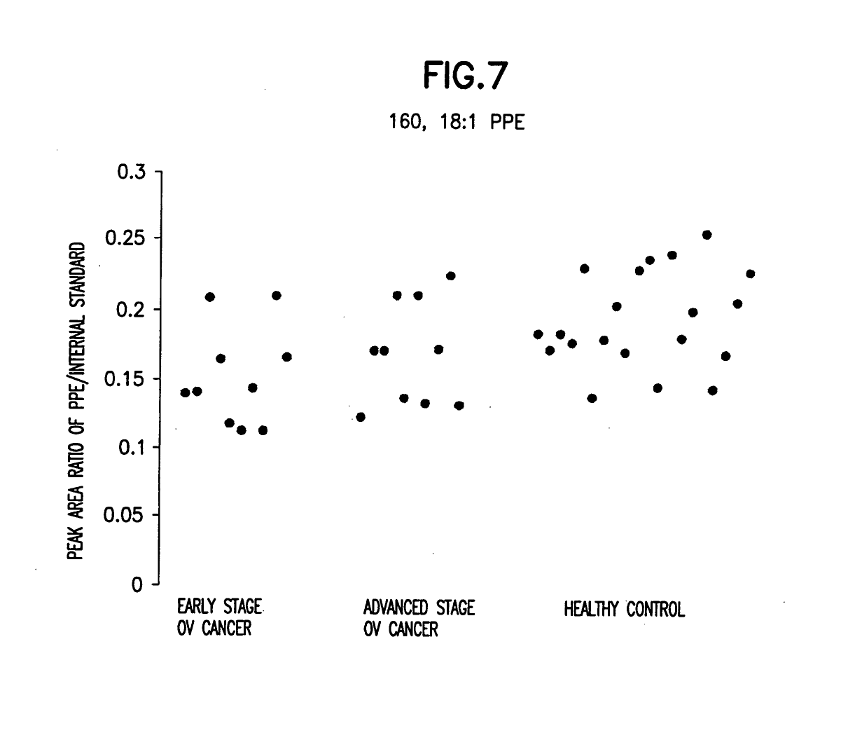

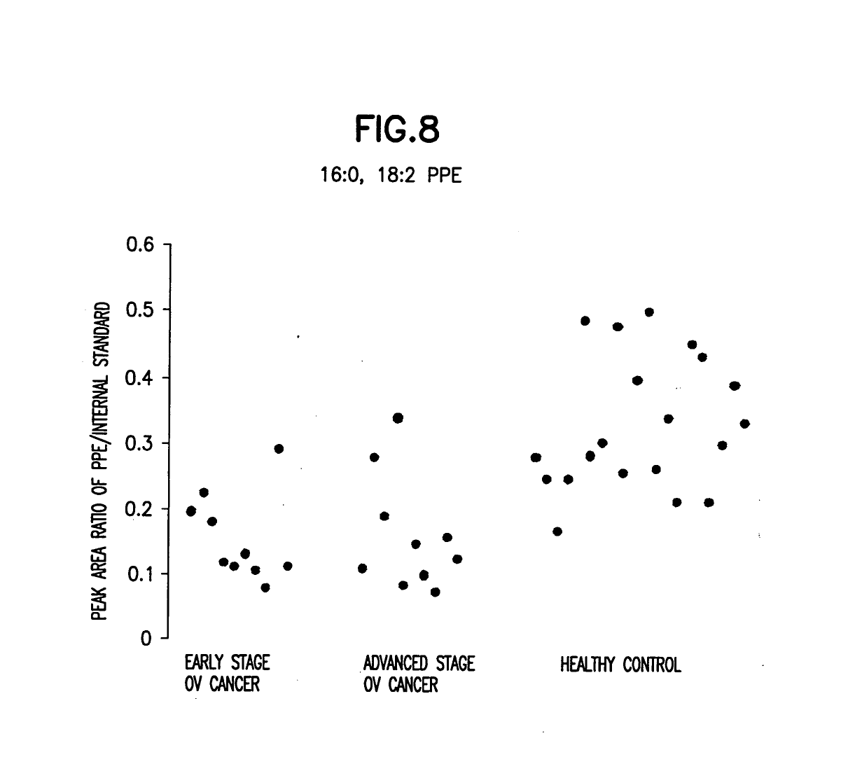

40 serum samples were collected. Among them were 10 early stage ovarian cancer, 10 late stage ovarian cancer, and 20 healthy control. Data analysis was done using the student t-test and the peak area ratio of analyte to internal standard was determined. The results are shown in Table 1 and FIG. 1 to FIG. 8.

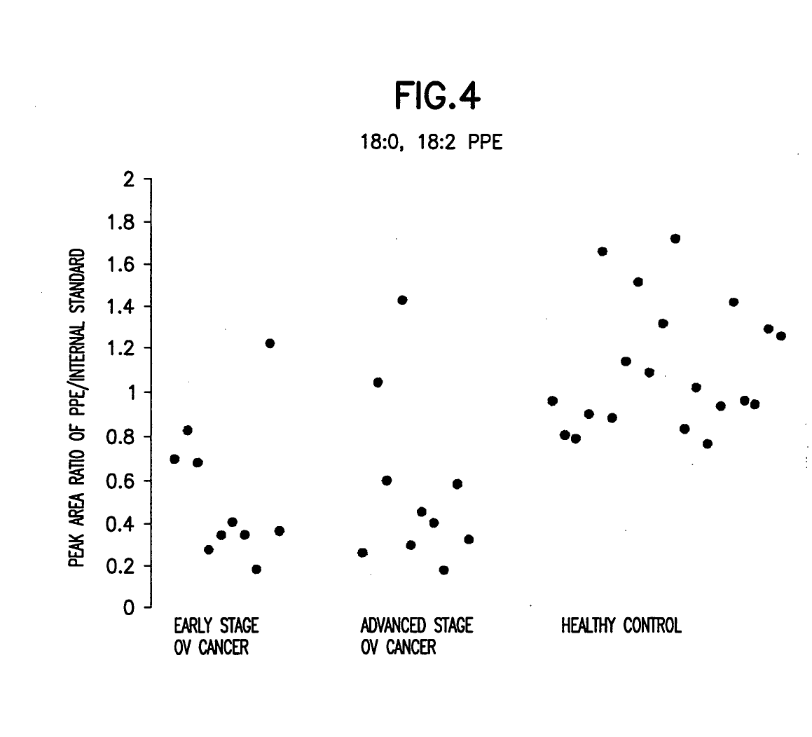

TABLE 1
Level of 18:0, 18:2 plasmenyl-PE (see FIG. 4), standard deviation,
and p value (related to healthy control samples) in 40 serum samples,
as determined by peak ratio of analyte to internal standard

| | Level of 18:0, 18:2 | Standard | |
|---|---|---|---|
| Serum sample | plasmenyl-PE | Deviation | p value |
| Early stage ovarian cancer | 0.537 | 0.322 | <0.001 |
| Advanced stage ovarian | 0.555 | 0.397 | <0.001 |
| cancer | | | |
| Healthy control | 1.11 | 0.298 | — |

If 0.70 is used as the cut-off, the levels of 18:0, 18:2 plasmenyl-PE in 8 of 10 early stage ovarian cancer patients are below this value, with the sensitivity equaling 80%. The levels of 18:0, 18:2 plasmenyl-PE in-8 of 10 advanced stage ovarian cancer are below this value, with the sensitivity equaling 80%. The levels of 16:0, 18:2 plasmenyl-PE in 10 of 10 healthy controls are above this value, with the specificity equaling 100%.

### Example 2

### Plasmenyl-PE in Plasma Samples

Materials

18:0, 22:6 PPE; 18:0, 20:4 PPE; 18:0, 18:1 PPE were purchased from Avanti Polar Lipids (Alabaster, Ala., USA). Using these lipids, it was determined that the MRM transition of 18:0, 22:6 PPE; 18:0, 20:4 PPE; 18:0, 18:1 PPE; 18:0, 18:2 PPE; 16:0 22:6 PPE; 16:0, 20:4 PPE; 16:0, 18:1 PPE; and 16:0, 18:2 PPE were 774.2→3.27.2, 750.2→303.2, 728.2→281.2, 726.2→279.2, 746.2→327.2, 722.2→303.2, 700.2→281.2, 698.2→279.2 respectively.

### Example 2(a)

### Extraction of Plasmenyl-PE from Plasma Samples

Lipid extraction was done according to the following procedure: Add 200 μL 10 μM 1,2-diheptadecanoyl-sn-glycerol-3-phosphoethanolamine, the internal standard for the assay, into 50 μl plasma samples. Vortex and add 2 ml 2:1 methanol-chloroform into the samples. Vortex again and centrifuge the mixture for 5 minutes at 4000 rpm and 10° C. Transfer the upper liquid layer into a test tube and dry the liquid layer under nitrogen. Then add 400 μl 0.1 M ammonium acetate in methanol into the nitrogen-dried lipids. Vortex and transfer everything in the test tube into a microcentrifuge tube. Centrifuge at 9000 rpm for 5 minutes. Transfer the supernatant into an injection vial for LC/ESI/MS/MS analysis.

### Example 2(b)

### MRM LC/ESI/MS/MS Analysis for Plasmenyl-PE

LC/ESI/MS/MS analysis of plasmenyl-PE species was performed using a Quatro micro mass spectrometer (Micromass, Altrincham, U.K.) equipped with an electrospray ionization (ESI) probe and interfaced with a Shimadzu SCL-10AvpHPLC system (Shimadzu, Tokyo, Japan). Lipids were separated with a Betabasic-18 column (20×2.1 mm, 5 μm, Thermo Electron, Waltham, Mass.), protected by a Betabasic 18 pre-column (10×2.1 mm, 5 μm, Thermo Electron, Waltham, Mass.). 300 μl ammonium phosphate, pH=5.46 buffer was used as mobile phase A while 9:1 (v:v) methanol-acetonitrile was used as mobile phase B. The gradient used was as follows: the column was first equilibrated with 70% B (30% A), followed by a linear change from 70% B (30% A) to 100% B (0% A) at 200 μl/minutes in the first 5 minutes. The gradient was kept at 100% in the following 3 minutes. Then it was changed back to 70% B (30% A) to re-equilibrate the column. The flow rate is 200 μl/minutes. Mass spectrometric analyses were performed online using electrospray ionization/tandem mass spectrometry in the negative multiple reaction monitoring (MRM) mode (capillary voltage: 3.5 KV, cone potential 55 V, collision energy 30 eV). The MRM transitions used have been described above in the section entitled “Materials” for Example 2.

### Example 2(c)

### Samples and Statistical Analysis

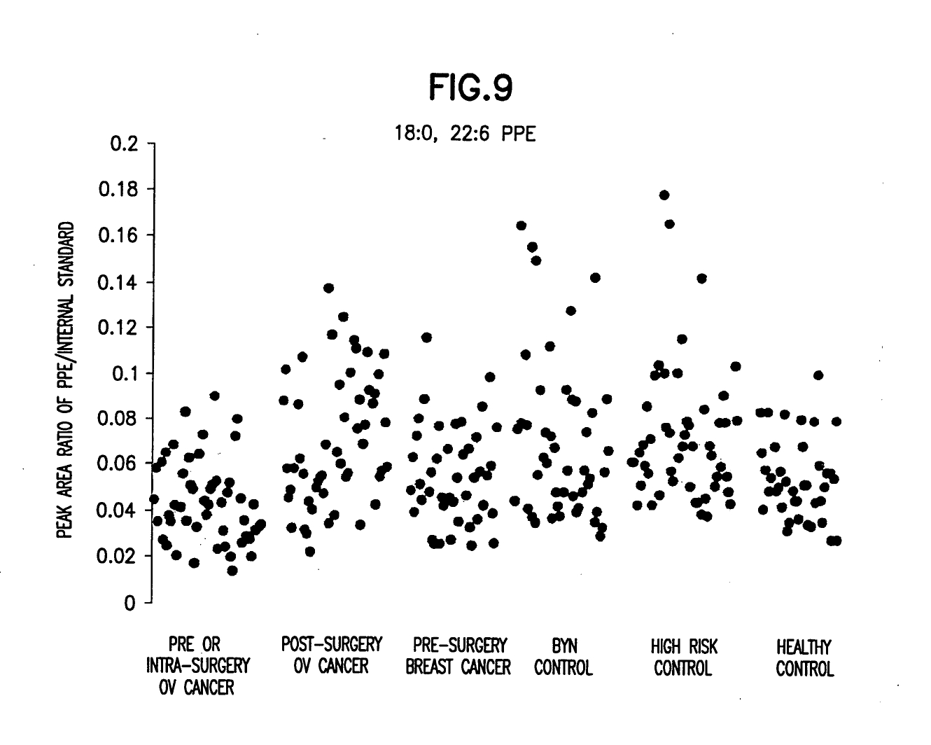

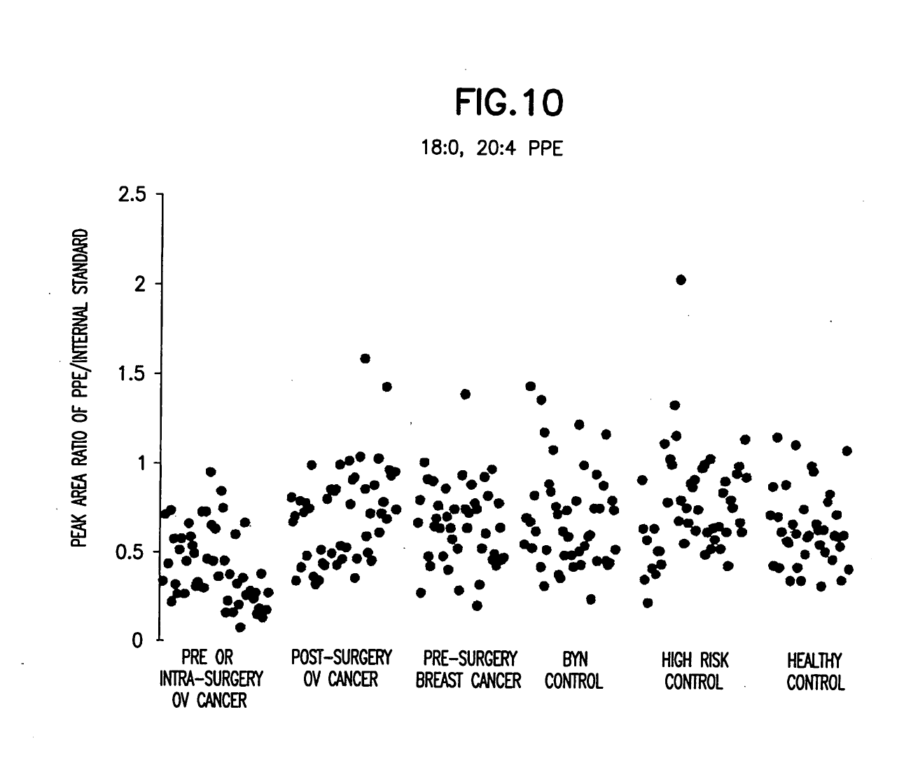

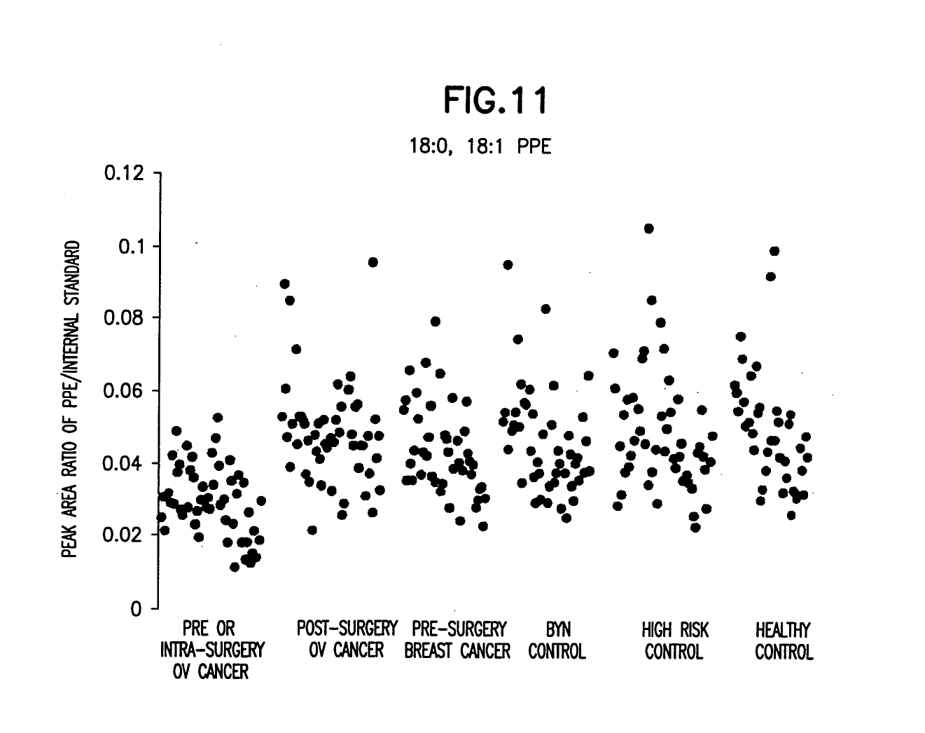

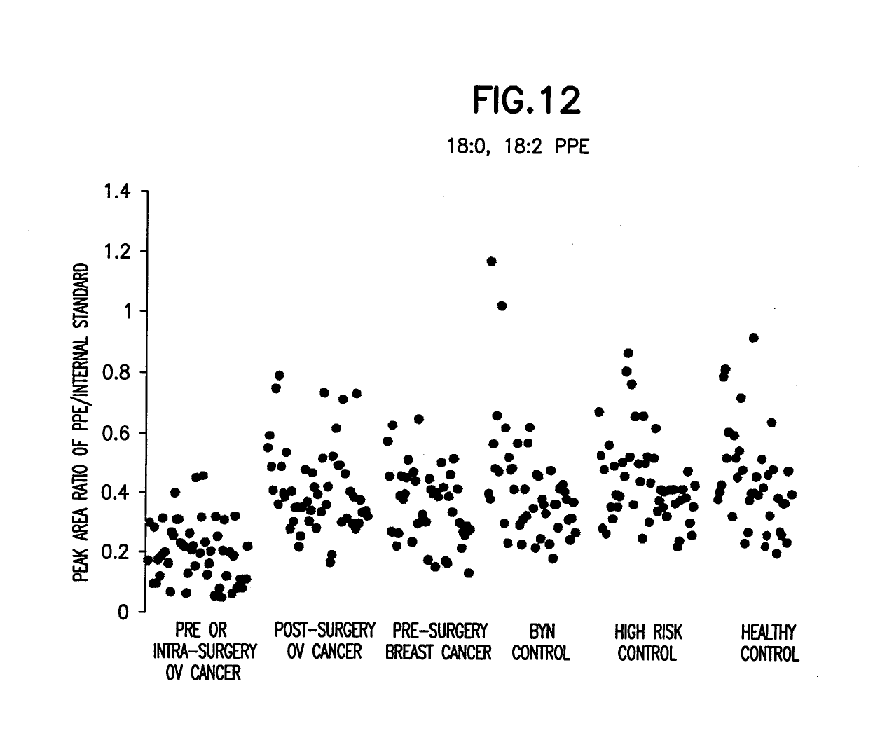

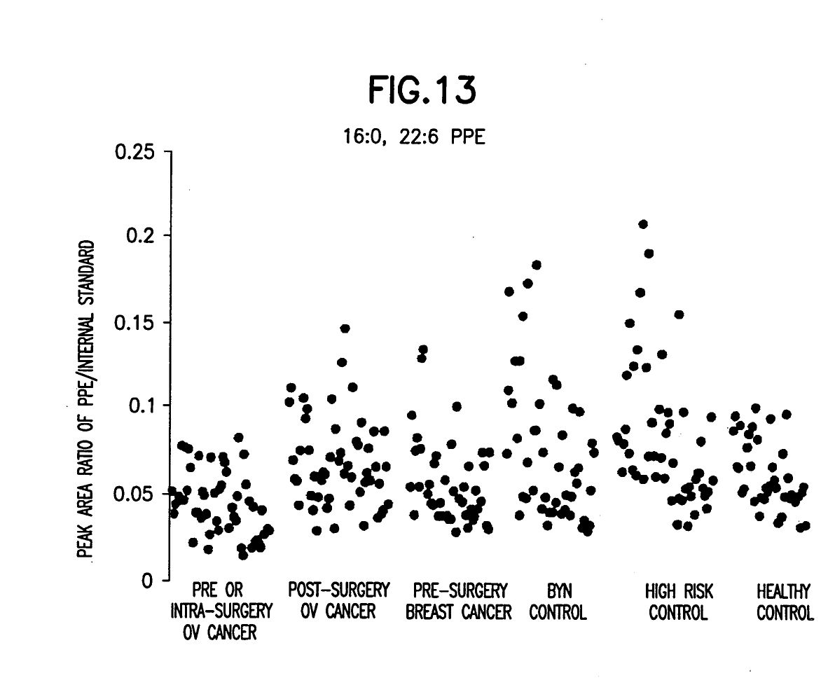

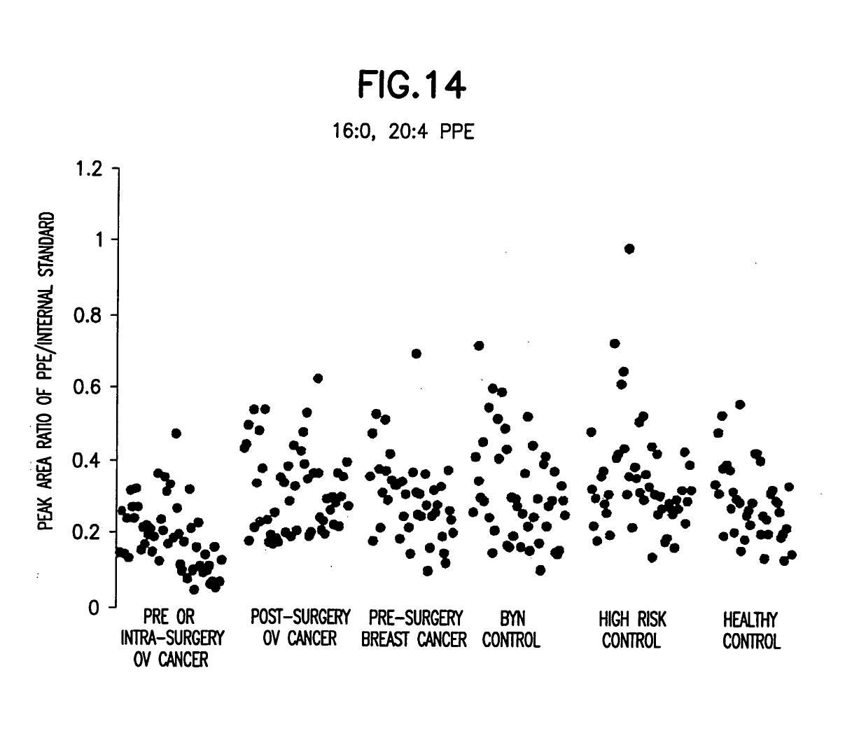

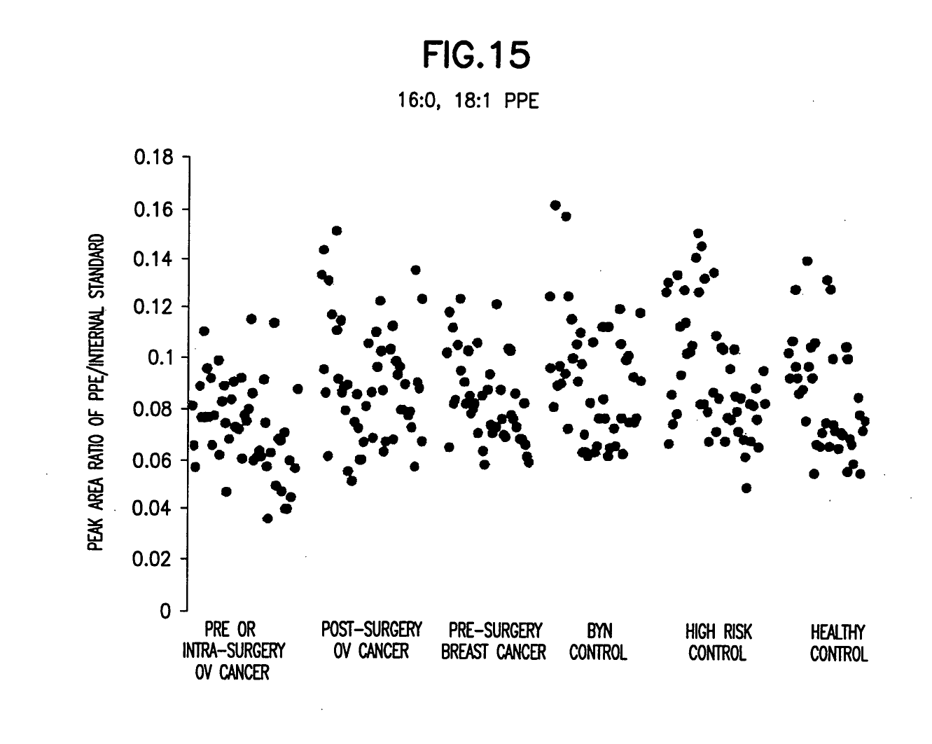

281 human plasma samples were collected in 10 different clinical sites. Among them were: 51 pre- or intra-surgery ovarian (“ov”) cancer, 52 post-surgery ov cancer, 43 pre-surgery breast cancer, 46 benign gynecological disease (“BYN”) control, 50 high-risk control and 39 healthy control. Data analysis was done using the student t-test and the peak area ratio of analyte to internal standard was determined. The results are shown in Table 2 and FIG. 9 to FIG. 16.

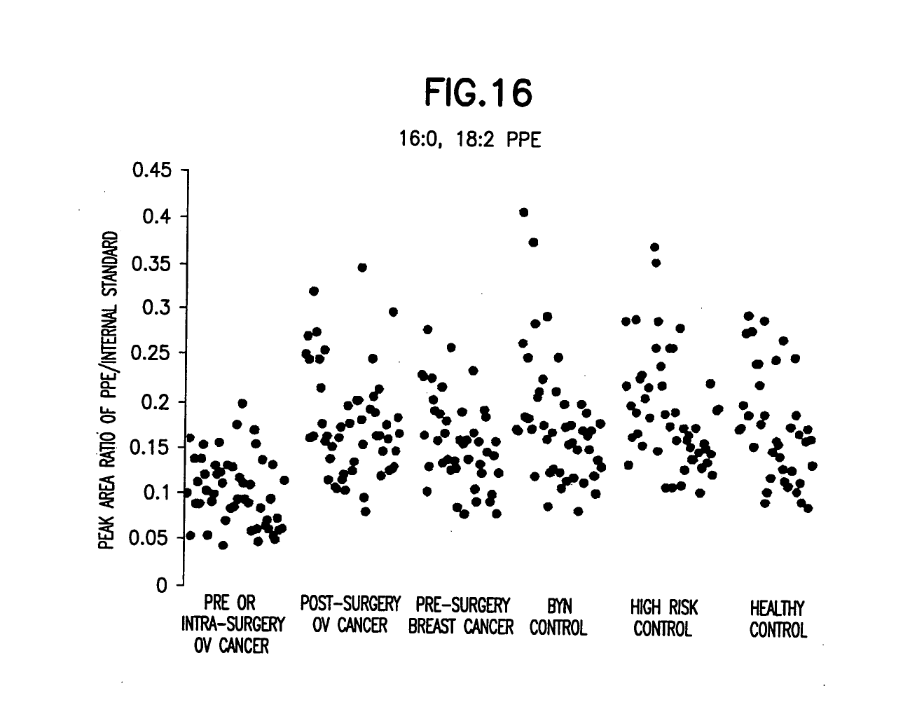

TABLE 2
Level of 16:0, 18:2 plasmenyl-PE (see FIG. 16), standard deviation,
and p value (related to pre- or intra-surgery ovarian cancer) in 281
plasma samples, as determined by the peak area ratio of analyte to
internal standard

| | Level of 18:0, 18:2 | Standard | |
|---|---|---|---|
| Plasma samples | plasmenyl-PE | Deviation | p value |
| Pre- or intra-surgery ov cancer | 0.102 | 0.038 | — |
| Post-surgery ov cancer | 0.175 | 0.059 | <0.001 |
| Pre-surgery breast cancer | 0.154 | 0.050 | <0.001 |
| Benign gynecological disease | 0.173 | 0.068 | <0.001 |
| (“BYN”) control | | | |
| High-risk control | 0.184 | 0.062 | <0.001 |
| Healthy control | 0.167 | 0.050 | <0.001 |

If 0.13 is used as the cutoff, 10 of 51 pre-surgery ovarian cancer are above the cutoff, with a sensitivity=80.4%. 11 of 43 breast cancer are below the cutoff, with a specificity=74.4%. 8 of 50 high risk control are below the cutoff, with a specificity=84%. 12 of 46 of benign gynecological disease control are below the cutoff, with a sensitivity=74.0%. 11 of 39 healthy control are below the cutoff, with a sensitivity=71.8%.

### Example 3(a)

### Preparation of Plasmenyl-PA From Plasmenyl-PC

0.6 μmol of plasmenyl-PC were placed into 90 μl of 50 mM Tris-HCl buffer, 10 mM CaCl₂, 1% triton (pH 8.0) and sonicated for 10 minutes. 5 μl of PLD enzyme (10 units, 1 unit will liberate 1.0 μM of choline from L-α-phosphatidylcholine (egg yolk) per hour at pH 5.6 at 30° C.) were added and the reaction was carried out for 4 hours at 37° C. The reaction was stopped by adding 0.4 ml of the extraction solvent, chloroform/methanol (2:1, v/v). A Bligh-Dyer extraction was performed and the organic phase and the aqueous phase were dried under N₂ respectively. See the following reaction scheme:

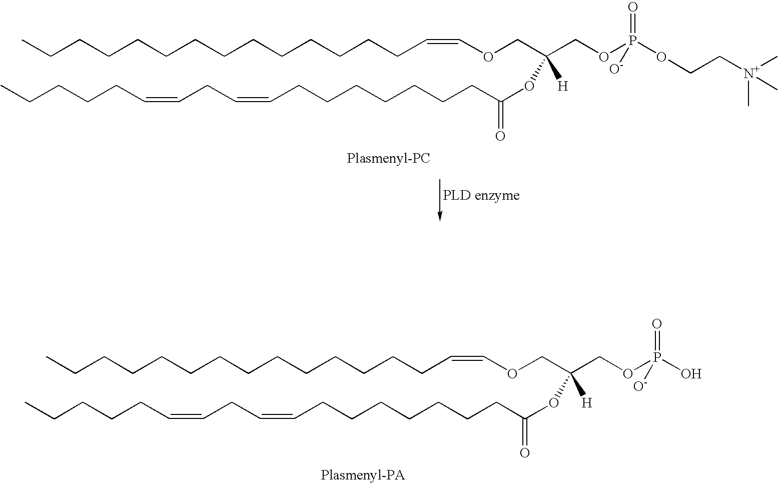

A crude plasmenyl-PA product was thus obtained and a mass spectrometric experiment was done to get the MRM (multiple reaction monitoring) transitions for plasmenyl-PA.

### Example 3(b)

### Extraction of Lipids From Plasma or Serum Samples

Lipid extraction was done according to the following procedure: Add 100 μl 2 μg/ml 1,2-diphytanoyl-sn-glycerol-3-phosphate, the internal standard for the assay, into 400 μl plasma or serum samples. Vortex and add 2 ml 2:1 methanol-chloroform into the samples. Vortex again and centrifuge the mixture for 5 minutes at 4000 rpm and 10° C. Transfer the upper liquid layer into a test tube and dry the liquid layer under nitrogen. Then add 200 μl 0.1 M ammonium acetate in methanol into the nitrogen-dried lipids. Vortex and transfer everything in the test tube into a microcentrifuge tube. Centrifuge at 9000 rpm for 5 minutes. Transfer the supernatant into an injection vial for MRM LC/ESI/MS/MS (liquid chromatography/electrospray ionization/tandem mass spectroscopy) analysis.

### Example 3(c)

### LC/ESI/MS/MS Analysis

MRM LC/ESI/MS/MS analysis of the plasmenyl-PA compound from Example 3(b) was performed using a Quatro micro mass spectrometer (Micromass, Altrincham, U.K.) equipped with an electrospray ionization (ESI) probe and interfaced with a Shimadzu SCL-10AvpHPLC system (Shimadzu, Tokyo, Japan). Lipids were separated with a Betabasic-18 column (20×2.1 mm, 5 μm, Thermo Electron, Waltham, Mass.), protected by a Betabasic-18 pre-column (10×2.1 mm, 5 μm, Thermo Electron, Waltham, Mass.). 300 μM ammonium phosphate, pH=5.46 buffer was used as mobile phase A, while 9:1 (v:v) methanol-acetonitrile was used as mobile phase B. The gradient used was as follows: the column was first equilibrated with 70% B (30% A), followed by a linear change from 70% B (30% A) to 100% B (0% A) at 200 μl/minutes in the first 5 minutes. The gradient was kept at 100% in the following 3 minutes. Then it was changed back to 70% B (30% A) to re-equilibrate the column. The flow rate was 200 μl/minutes. Mass spectrometric analyses were performed online using electrospray ionization/tandem mass spectrometry in the negative multiple reaction monitoring (MRM) mode (capillary voltage: 3.0 KV; cone potential: 55 V; collision energy: 25 eV). The MRM transitions used to detect plasmenyl-PA were the mass charge ratio for the molecular anion M⁻ and its daughter ion (m/z of 375.2).

What is claimed is:

1. A method of detecting an inflammatory disease in a test subject comprising:
(a) determining the amount of plasmenyl-PE in a sample of a bodily fluid taken from the test subject, and
(b) comparing the amount of plasmenyl-PE in the sample of the bodily fluid taken from the test subject to a range of amounts of plasmenyl-PE found in samples of said bodily fluid taken from a group of normal subjects of the same species as the test subject and lacking the inflammatory disease, whereby a change in the amount of the plasmenyl-PE in the sample of the bodily fluid taken from the test subject indicates the presence of an inflammatory disease.

2. The method of claim 1, wherein the test subject is a human.

3. The method of claim 2, wherein, in step (b), the change in the amount is a lower amount.

4. A method of detecting an inflammatory disease in a test subject comprising:
(a) determining the amount of a biomarker having a mass charge ratio of approximately 698.2, 722.2, 726.2 or 750.2 in a sample of a bodily fluid taken from the test subject, and
(b) comparing the amount of the biomarker in the sample of the bodily fluid taken from the test subject to a range of amounts of the biomarker found in the samples of the bodily fluid taken from a group of normal subjects of the same species as the test subject and lacking the inflammatory disease, whereby a change in the amount of the biomarker in the sample of the bodily fluid taken from the test subject indicates the presence of the inflammatory disease.

5. The method of claim 4, wherein the test subject is a human.

6. The method of claim 5, wherein the change in the amount is a lower amount.

7. A method of detecting a cancer in a test subject comprising:
(a) determining the amount of plasmenyl-PE in a sample of a bodily fluid taken from the test subject, and
(b) comparing the amount of plasmenyl-PE in the sample of the bodily fluid from the test subject to a range of amounts of plasmenyl-PE found in the samples of said bodily fluids taken from a group of normal subjects of the same species as the test subject and lacking the cancer, whereby a change in the amount of the plasmenyl-PE in the sample of the bodily fluid from the test subject indicates the presence of the cancer.

8. The method of claim 7, wherein the test subject is a human.

9. The method of claim 8, wherein, in step (b), the change in the amount is a lower amount.

10. The method of claim 9, wherein the cancer is ovarian cancer.

11. The method of claim 10, wherein the bodily fluid is serum or plasma.

12. The method of claim 11, wherein the plasmenyl-PE is selected from the group consisting of 16:0, 18:2 PPE; 18:0, 22:6 PPE; 18:0, 20:4 PPE; 16:0, 22:6 PPE; 18:0, 18:1 PPE; 18:0, 18:2 PPE; 16:0, 20:4 PPE and 16:0, 18:1 PPE.

13. The method of claim 10, wherein the plasmenyl-PE is 18:0, 18:2 PPE.

14. The method of claim 10, wherein the plasmenyl-PE is 18:0, 20:4 PPE.

15. The method of claim 10, wherein the plasmenyl-PE is 16:0, 18:2 PPE.

16. The method of claim 10, wherein the plasmenyl-PE is 16:0, 20:4 PPE.

17. A method of detecting a cancer in a test subject comprising:
(a) determining the amount of a biomarker having a mass charge ratio of approximately 698.2, 722.2, 726.2 or 750.2 in a sample of a bodily fluid taken from the test subject, and
(b) comparing the amount of the biomarker in the sample of the bodily fluid from the test subject to a range of amounts of the biomarker found in samples of said bodily fluid from a group of normal subjects of the same species as the test subject and lacking the cancer, whereby a change in the amount of the biomarker in the sample of the bodily fluid from the test subject indicates the presence of cancer.

18. The method of claim 17, wherein the test subject is a human.

19. The method of claim 18, wherein, in step (b), the change in the amount is a lower amount.

20. The method of claim 18, wherein the cancer is ovarian cancer.

21. The method of claim 20, wherein the bodily fluid is serum.

22. The method of claim 20, wherein the bodily fluid is plasma.

23. A method for detecting the occurrence of ovulation during a menstrual cycle in a female test subject comprising:
(a) determining the amount of plasmenyl-PE in a sample of a bodily fluid taken from a female test subject, and
(b) comparing the amount of plasmenyl-PE in the sample of the bodily fluid taken from the female test subject to a range of amounts of plasmenyl-PE found in samples of said bodily fluid from a group of non-ovulating female subjects of the same species as the test subject, whereby a change in the amount of the plasmenyl-PE in the sample of the bodily fluid from the female test subject indicates the occurrence of ovulation.

24. The method of claim 23, wherein the test subject is a human.

25. The method of claim 24, wherein, in step (b), the change in the amount is a lower amount.

26. A method of detecting the occurrence of ovulation during a menstrual cycle in a female test subject comprising:
(a) determining the amount of a biomarker having a mass charge ratio of approximately 698.2, 722.2, 726.2 or 750.2 in a sample of a bodily fluid taken from the female test subject, and
(b) comparing the amount of the biomarker in the sample of the bodily fluid taken from the female test subject to a range of amounts of the biomarker found in samples of said bodily fluid from a non-ovulating female subject of the same species as the test subject, whereby a change in the amount of the biomarker in the sample of the bodily fluid from the female test subject indicates the occurrence of ovulation.

27. The method of claim 26, wherein the test subject is a human.

28. The method of claim 27, wherein, in step (b), the change in the amount is a lower amount.

29. A method for monitoring the presence of an inflammatory disease in a test subject over time comprising:
(a) determining the amount of plasmenyl-PE in a sample of a bodily fluid taken from the test subject at a first time,
(b) determining the amount of plasmenyl-PE in a sample of the bodily fluid taken from said test subject at a second time, which is later than the first time,
(c) comparing the amounts of plasmenyl-PE in step (a) and step (b) to determine whether there has been an increase or a decrease in the amount of plasmenyl-PE in the sample of the bodily fluid taken from the test subject at the later time relative to the amount of the plasmenyl-PE in the sample taken from the test subject at the first time, whereby a decrease in the amount of plasmenyl-PE in the sample of the bodily fluid at the later time indicates the presence of, or worsening of, the inflammatory disease, or an increase in the amount of plasmenyl-PE in the sample of the bodily fluid at the later time indicates an absence, or improvement of, the inflammatory disease.

30. The method of claim 29, wherein the test subject is a human.

31. A method for monitoring an inflammatory disease in a test subject over time comprising:
(a) determining the amount of a biomarker having a mass charge ratio of approximately 698.2, 722.2, 726.2 or 750.2 in a sample of a bodily fluid taken from the test subject at a first time,
(b) determining the amount of the biomarker in a sample of a bodily fluid taken from said test subject at a second time, which is later than the first time,
(c) comparing the amounts of the biomarker in step (a) and step (b) to determine whether there has been an increase or a decrease in the amount of the biomarker in the sample of the bodily fluid taken from the test subject at the later time relative to the amount of the biomarker in the sample of the bodily fluid taken from the test subject at the first time, whereby a decrease in the amount of the biomarker in the sample of the bodily fluid at the later time indicates the presence of, or worsening of, the inflammatory disease, or an increase in the amount of the biomarker in the sample of the bodily fluid at the later time indicates an absence, or improvement of, the inflammatory disease.

32. The method of claim 31, wherein the test subject is a human.

33. A method for monitoring a cancer in a test subject over time comprising:
(a) determining the amount of plasmenyl-PE in a sample of a bodily fluid taken from the test subject at a first time,
(b) determining the amount of plasmenyl-PE in a sample of the bodily fluid taken from said test subject at a second time, which is later than the first time,
(c) comparing the amounts of plasmenyl-PE in step (a) and step (b) to determine whether there has been an increase or a decrease in the amount of plasmenyl-PE in the sample of the bodily fluid taken from the test subject at the later time relative to the amount of the plasmenyl-PE in the sample of the bodily fluid taken from the test subject at the first time, whereby a decrease in the amount of the plasmenyl-PE in the sample of the bodily fluid at the later time indicates the presence of, or worsening of, the cancer, or an increase in the amount of the plasmenyl-PE in the sample of the bodily fluid at the later time indicates an absence, or improvement of, the cancer.

34. The method of claim 33, wherein the test subject is a human.

35. The method of claim 34, wherein the cancer is ovarian cancer.

36. The method of claim 34, wherein the plasmenyl-PE is selected from the group consisting of 16:0, 18:2 PPE; 18:0, 22:6 PPE; 18:0, 20:4 PPE, 16:0, 22:6 PPE; 18:0, 18:1 PPE; 18:0, 18:2 PPE; 16:0, 20:4 PPE; and 16:0, 18:1 PPE.

37. The method of claim 33, wherein the plasmenyl-PE is selected from the group consisting of 18:0, 18:2 PPE, 18:0, 20:4 PPE, 16:0, 18:2 PPE and 16:0, 20:4 PPE.

38. A method for monitoring a cancer in a test subject over time comprising:
(a) determining the amount of a biomarker having a mass charge ratio of approximately 698.2, 722.2, 726.2 or 750.2 of a bodily fluid taken from the test subject at a first time,
(b) determining the amount of the biomarker in a sample of the bodily fluid taken from said test subject at a second time, which is later than the first time,
(c) comparing the amounts of the biomarker in step (a) and step (b) to determine whether there has been an increase or a decrease in the amount of the biomarker in the sample of the bodily fluid taken from the test subject at the later time relative to the amount of the biomarker in the sample of the bodily fluid taken from the test subject at the first time, whereby a decrease in the amount of the biomarker in the sample of the bodily fluid at the later time indicates the presence of, or worsening of, the cancer, or an increase in the amount of the biomarker in the sample of the bodily fluid at the later time indicates an absence, or improvement of, the cancer.

39. The method of claim 38, wherein the test subject is a human.

40. The method of claim 39, wherein the cancer is ovarian cancer.

41. The method of claim 40, wherein the bodily fluid is serum.

42. A method of detecting an inflammatory disease in a test subject comprising:
(a) determining the amount of a plasmalogen in a sample of a bodily fluid taken from the test subject, and
(b) comparing the amount of the plasmalogen in the sample of the bodily fluid taken from the test subject to a range of amounts of the plasmalogen found in samples of said bodily fluid taken from a group of normal subjects of the same species as the test subject and lacking the inflammatory disease, whereby a change in the amount of the plasmalogen in the sample of the bodily fluid taken from the test subject indicates the presence of an inflammatory disease.

43. A method of detecting a cancer in a test subject comprising:
(a) determining the amount of a plasmalogen in a sample of a bodily fluid taken from the test subject, and
(b) comparing the amount of the plasmalogen in the sample of the bodily fluid taken from the test subject to a range of amounts of the plasmalogen found in the samples of said bodily fluids taken from a group of normal subjects of the same species as the test subject and lacking the cancer, whereby a change in the amount of the plasmalogen in the sample of the bodily fluid taken from the test subject indicates the presence of the cancer,
wherein when the cancer is ovarian cancer, the plasmalogen is not PPA or PPC, and wherein when the cancer is breast cancer, the plasmalogen is not PPE or PPA.

44. A method for detecting the occurrence of ovulation during a menstrual cycle in a female test subject comprising:
(a) determining the amount of a plasmalogen in a sample of a bodily fluid taken from a female test subject, and
(b) comparing the amount of the plasmalogen in the sample of the bodily fluid taken from the female test subject to a range of amounts of the plasmalogen found in samples of said bodily fluid taken from a group of non-ovulating female subjects of the same species as the test subject, whereby a change in the amount of the plasmalogen in the sample of the bodily fluid taken from the female test subject indicates the occurrence of ovulation.

45. A method for monitoring the presence of an inflammatory disease in a test subject over time comprising:
(a) determining the amount of a plasmalogen in a sample of a bodily fluid taken from the test subject at a first time,
(b) determining the amount of the plasmalogen in a sample of the bodily fluid taken from said test subject at a second time, which is later than the first time,
(c) comparing the amounts of the plasmalogen in step (a) and step (b) to determine whether there has been an increase or a decrease in the amount of the plasmalogen in the sample of the bodily fluid taken from the test subject at the later time relative to the amount of the plasmalogen in the sample taken from the test subject at the first time, whereby a decrease in the amount of the plasmalogen in the sample of the bodily fluid at the later time indicates the presence of, or worsening of, the inflammatory disease, or an increase in the amount of the plasmalogen in the sample of the bodily fluid at the later time indicates an absence, or improvement of, the inflammatory disease.

46. A method for monitoring a cancer in a test subject over time comprising:
(a) determining the amount of a plasmalogen in a sample of a bodily fluid taken from the test subject at a first time,
(b) determining the amount of the plasmalogen in a sample of the bodily fluid taken from said test subject at a second time, which is later than the first time,
(c) comparing the amounts of the plasmalogen in step (a) and step (b) to determine whether there has been an increase or a decrease in the amount of the plasmalogen in the sample of the bodily fluid taken from the test subject at the later time relative to the amount of the plasmalogen in the sample of the bodily fluid taken from the test subject at the first time, whereby a decrease in the amount of the plasmalogen in the sample of the bodily fluid at the later time indicates the presence of or worsening of the cancer, or an increase in the amount of the plasmalogen in the sample of the bodily fluid at the later time indicates an absence, or improvement of, the cancer,
wherein when the cancer is ovarian cancer, the plasmalogen is not PPA or PPC, and wherein when the cancer is breast cancer, the plasmalogen is not PPE or PPA.
